(19) United States
(12) Patent Application Publication
(10) Pub. No.: US 2013/0143866 A1
(43) Pub. Date: Jun. 6, 2013

(54) ANTICANCER COMBINATION TREATMENTS

(71) Applicant: Paul W. Sylvester, West Monroe, LA (US)

(72) Inventor: Paul W. Sylvester, West Monroe, LA (US)

(21) Appl. No.: 13/691,794

(22) Filed: Dec. 2, 2012

Related U.S. Application Data
(60) Provisional application No. 61/566,299, filed on Dec. 2, 2011.

(51) Int. Cl. A61K 31/553; A61K 31/404; A61K 31/355; A61K 31/496; A61K 31/519; A61K 31/475; A61K 31/5377; A61K 31/5025; A61K 31/045

(52) U.S. Cl. CPC A61K 31/553; A61K 31/355; A61K 31/496; A61K 31/404; A61K 31/475; A61K 31/5377; A61K 31/5025; A61K 31/519
USPC 514/211.08; 514/254.09; 514/414; 514/294; 514/235.2; 514/248; 514/252.16

(57) ABSTRACT

Compositions of matter including γ-tocotrienol and SU11274 are disclosed herein. A variety of Met kinase inhibitors may be used in conjunction with tocotrienols in the treatment or prevention of cancer. Combinations of γ-tocotrienol and SU11274 inhibit the cell growth of +SA mammary tumor cells.

This application claims the benefit of U.S. Provisional Application No. 61/566,299 filed Dec. 2, 2011.

In cancer cells, constitutive activation of Met, which can occur through mutation, overexpression, and/or interaction with other cell surface receptors such as members within the HER/ErbB family of receptor tyrosine kinases, can result in unregulated Met activation and greatly enhanced downstream signaling. Dysregulation of Met signaling can ultimately lead to enhanced tumor cell proliferation, resistance to apoptosis, angiogenesis, invasion, and metastasis. Compositions and treatments disclosed herein have the potential to impact Met, regulate tumor cell growth and influence many other tumor cell properties.

### BRIEF DESCRIPTION OF THE DRAWINGS

FIG. 1 shows +SA mammary tumor cell growth in response to various doses of HGF over a 3-day culture period.

FIG. 2 shows the effects of various doses of SU11274, a specific Met Receptor Tyrosine Kinase inhibitor, or γ-tocotrienol on HGF-dependent +SA mammary tumor cell growth after a 3-day culture period.

FIG. 3 shows the effects of 5.5 μM SU11274 treatment or 4 μM γ-tocotrienol treatment on HGF-dependent Met receptor expression and activation using Western blot images.

FIG. 4 shows the time dependent inhibitory effect of γ-tocotrienol treatment on Met receptors using Western blot images.

FIG. 5 shows cell counts after treatment with subeffective doses γ-tocotrienol and SU11274 both independently and together on HGF-dependent growth of +SA mammary tumor cells.

FIG. 6 shows the effects of SU11274 or γ-tocotrienol treatment on HGF-dependent expression and tyrosine phosphorylation (activation) of Met in +SA mammary tumor cells using Western blot images.

### DETAILED DESCRIPTION

+SA mammary tumor cells were used for the examples presented herein. The +SA mammary tumor cell line is characterized as being highly malignant, estrogen-independent, and displays anchorage-independent growth when cultured in soft agarose gels. In addition, +SA cells can form adenocarcinomas that metastasize to the lungs when injected into the mammary gland fat pad of syngeneic female mice. +SA cells were maintained in serum-free defined media containing 10 ng/mL Hepatocyte growth factor (HGF) as the mitogen, Dulbecco's Modified Eagle's Medium (DMEM)/F12 supplemented with 5 mg/mL bovine serum albumin (BSA), 10 μg/mL transferrin, 100 U/mL soybean trypsin inhibitor, 100 U/mL penicillin, 0.1 mg/mL streptomycin and 10 μg/mL insulin at 37° C. in an environment of 95% air and 5% CO₂ in humidified incubator. For subculturing, cells were rinsed with sterile Ca²⁺ and Mg²⁺-free phosphate buffered saline (PBS) and incubated with 0.05% trypsin containing 0.025% EDTA in PBS for 5 min at 37° C. The released cells were centrifuged, resuspended in serum-free media and counted using hemocytometer.

γ-tocotrienol was prepared for the tests of this example by suspending it in a solution of sterile 10% BSA. An appropriate amount of γ-tocotrienol was dissolved in 100 μL of absolute ethyl alcohol and then added to a small volume of sterile 10% BSA in water and incubated overnight at 37° C. This stock solution was used to prepare various concentrations of the treatment media. Ethanol was added to all treatment groups in a given experiment such that the final concentration of ethanol never exceeded 0.1%. Stock solution of the Met inhibitor, SU11274 was prepared in DMSO and DMSO was added to all treatment media such that the final concentration was same in all treatment groups and never exceeded a concentration of 0.1%.

+SA cells were initially seeded at a density of 5×10⁴ cells/well (6 replicates/group) in 24-well plates in serum-free defined media containing 10 ng/mL HGF as mitogen and allowed to attach overnight. For growth studies, +SA cells were maintained in serum-free defined media containing various doses of HGF over a 3-day culture period. In the antiproliferative experiments, cells were exposed to respective experimental treatments containing various concentrations of SU11274 or γ-tocotrienol alone or in combination for three days. On the fourth day, cells were divided into different treatment groups and treated with various concentrations of SU11274 or γ-tocotrienol alone or in combination for 24 hours. At the end of the treatment exposure period, the viable +SA cell number was determined by 3-(4,5-dimethylthiazol-2yl)-2,5-diphenyl tetrazolium bromide (MTT) colorimetric assay. Control and treatment media in various treatment groups in 24-well plates were replaced with fresh media containing 0.41 mg/ml MTT. After 4-h incubation period, the media was removed and MTT crystals were dissolved in isopropanol (1 ml/well), and the optical density of each sample was measured at 570 nm on a microplate reader (SpectraCount, Packard BioScience Company). The number of cells/well was calculated against a standard curve prepared by plating various concentrations of cells, as determined by hemocytometer, at the beginning of each experiment.

### Example 1

### HGF Influenced Cell Growth

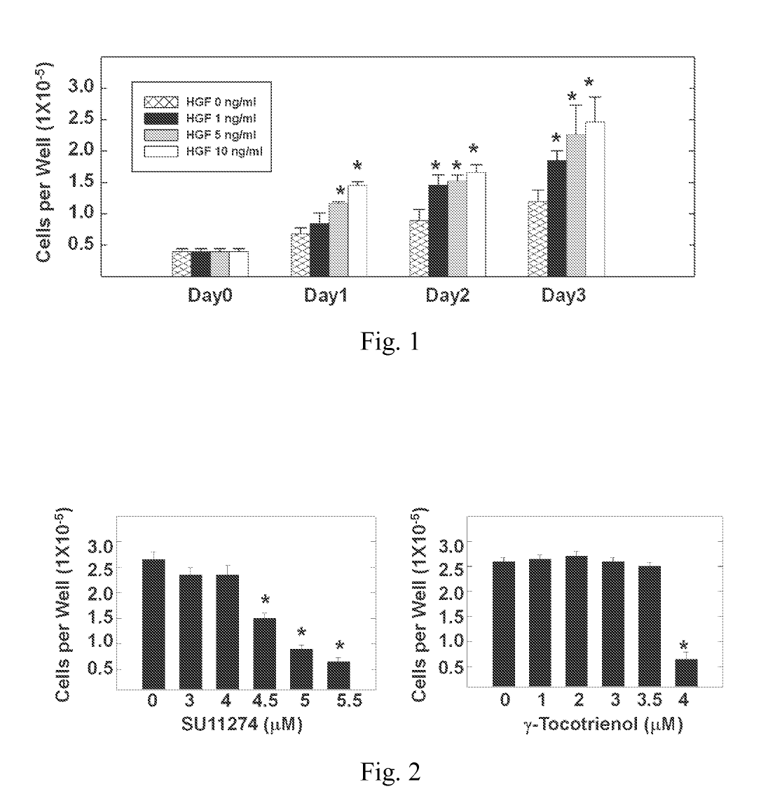

FIG. 1 shows +SA mammary tumor cell growth in response to various doses of HGF over a 3-day culture period. Stimulation with 1-10 ng/mL HGF significantly increased the growth of neoplastic +SA mammary epithelial cells in a dose-responsive manner as compared to cells maintained in mitogen-free control media. Vertical bars indicate mean viable cell number±SEM for six replicates in each treatment group. Differences among various treatment groups in +SA cell growth studies were determined by the analysis of variance (ANOVA) followed by Dunnett's t test. A difference of P<0.05 was considered statistically significant as compared to the vehicle-treated control.

### Example 2

### Single Treatment Dose Response

Effects of various doses of SU11274, a specific Met Receptor Tyrosine Kinase inhibitor, or γ-tocotrienol on HGF-dependent +SA mammary tumor cell growth after a 3-day culture period are shown in FIG. 2. Treatment with 4.5-5.5 μM SU11274 or 4 μM γ-tocotrienol significantly inhibited HGF-dependent +SA cell growth as compared to the cells in the vehicle-treated control group. In the present example, +SA cells were initially plated at a density of 5×10⁴ cells/well (6 replicates/group) in 24-well plates and exposed to various doses of SU11274 or γ-tocotrienol in the presence of 10 ng/mL HGF throughout a 3-day culture period. Viable cell number was determined using the MTT colorimetric assay. Vertical bars indicate the mean viable cell number±SEM in each treatment group. A difference of P<0.05 was considered statistically significant as compared to the vehicle-treated control.

### Example 3

### Impacts on Total Met and Phosphorylated Met

For Western blot analysis, +SA cells were initially plated at a density of 1×10⁶ cells/100-mm culture dish, allowed to attach overnight and then incubated in the respective control or treatment media. At the end of treatment period, cells were isolated with trypsin, washed with PBS, and then whole cell lysates were prepared in a Laemmle buffer. Protein concentration in each sample was determined using a protein assay kit (Bio-Rad protein assay kit, Bio Rad, Hercules, Calif., USA). Equal amount of protein (30-50 μg/lane) of each sample was subjected to electrophoresis through 7.5%-15% SDS-polyacrylamide minigels. Minigels within a given treatment were run simultaneously using AccuPower model 500 power supply unit (VWR, Suwanee, Ga., USA). Each gel was then equilibrated in transfer buffer and transblotted at 30 V for 12-16 hours at 4° C. onto a polyvinylidene fluoride (PVDF) membrane (PerkinElmer Lifesciences, Wellesley, Mass., USA) in a Trans-Blot Cell (Bio-Rad Laboratories, Hercules, Calif.) according to the method of Towbin. These PVDF membranes were then blocked with 2% BSA in 10 mM Tris-HCl containing 50 mM NaCl and 0.1% Tween 20, pH 7.4 (TBST) and then, incubated with specific primary antibodies against Met, phospho-Met, cleaved caspase-3, cleaved PARP, and α-tubulin diluted 1:1000 to 1:5000 in 2% BSA in TBST for 2 hours at room temperature or overnight at 4° C. Membranes were then washed 5 times with TBST and incubated with respective horseradish peroxide-conjugated secondary antibody diluted 1:3000 to 1:5000 in 2% BSA in TBST for 1 hour at room temperature followed by rinsing with TBST for 5 times. Blots were then visualized by chemiluminescence. (Pierce, Rockford, Ill., USA). Images of protein bands from all treatment groups within a given experiment were acquired using Kodak Gel Logic 1500 Imaging System (Carestream Health Inc, New Haven, Conn., USA). The visualization of α-tubulin was used to ensure equal sample loading in each lane. All experiments were repeated at least three times and a representative Western blot images displayed in the figures are representative of the overall results.

+SA cells were plated at a density of 1×10⁶ cells/100 mm culture dish. Cells were then incubated with serum-free media containing vehicle (control), 5.5 μM SU11274 or 4 μM γ-tocotrienol and 10 ng/mL HGF as the mitogen for a 3-day culture period. Following treatment exposure, cells were incubated in mitogen-free media overnight. Afterwards, cells were stimulated with 100 ng/mL HGF for 30 min Cells incubated in mitogen-free media were used as untreated controls. Following mitogen exposure, whole cell lysates were prepared and then subjected to polyacrylamide gel electrophoresis and Western blot analysis for total and phosphorylated Met levels. α-Tubulin was visualized to ensure equal sample loading in each lane. Each Western blot is a representative image of the data obtained for experiments that were repeated at least three times.

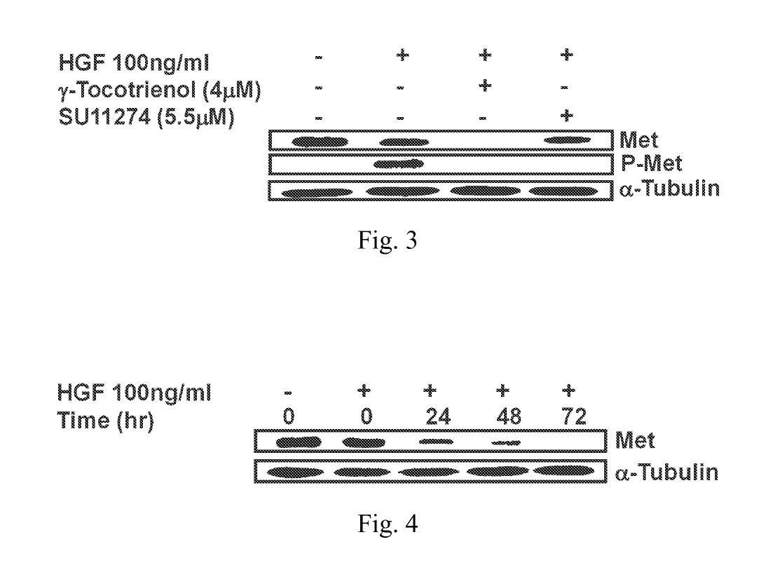

The effects of 5.5 μM SU11274 treatment or 4 μM γ-tocotrienol treatment on HGF-dependent Met receptor expression and activation is shown in FIG. 3. Western blot analysis showed that treatment with growth inhibitory concentration of SU11274 (5.5 μM) resulted in a large relative reduction in HGF-dependent Met tyrosine phosphorylation (activation), but had no effect on total intracellular levels of Met receptors as compared with +SA cells maintained in their respective vehicle-treated control groups. Alternatively, effective treatment of γ-tocotrienol (4 μM) caused a significant reduction in the level of total Met protein in +SA cells as shown in the Western blot images (FIG. 3). The loss of Met expression caused by γ-tocotrienol treatment mirrors the loss of HGF-induced Met phosphorylation.

### Example 4

### Met Dependence on γ-Tocotrienol Over Time

FIG. 4 shows that the inhibitory effect of γ-tocotrienol treatment on Met receptors is following a time-dependent manner. Effective treatment with 4 μM γ-tocotrienol resulted in a reduction in the total levels of Met protein in +SA cells started after 24 hours of treatment exposure and was maximal after 3-day of culture exposure to treatment as compared to their respective vehicle-treated control groups.

The +SA cells were plated at a density of 1×10⁶ cells/100 mm culture dish. Cells were then incubated with serum-free media containing vehicle (control) or 4 μM γ-tocotrienol, and 10 ng/mL HGF as a mitogen. Cells were collected at various times during the next 72-h period. Following treatment exposure at each time point, cells were incubated in mitogen-free media overnight. Afterwards, cells were stimulated with 100 ng/mL HGF for 30 min Cells incubated with mitogen-free media were used as untreated controls. Following mitogen exposure, whole cell lysates were prepared and then subjected to polyacrylamide gel electrophoresis and Western blot analysis for total and phosphorylated (active) Met receptor levels. α-Tubulin was visualized to ensure equal sample loading in each lane. Western blots presented in FIG. 4 are representative of the data obtained for experiments that were repeated at least three times.

### Example 5

### Synergystic Effect of γ-Tocotrienol with SU11274

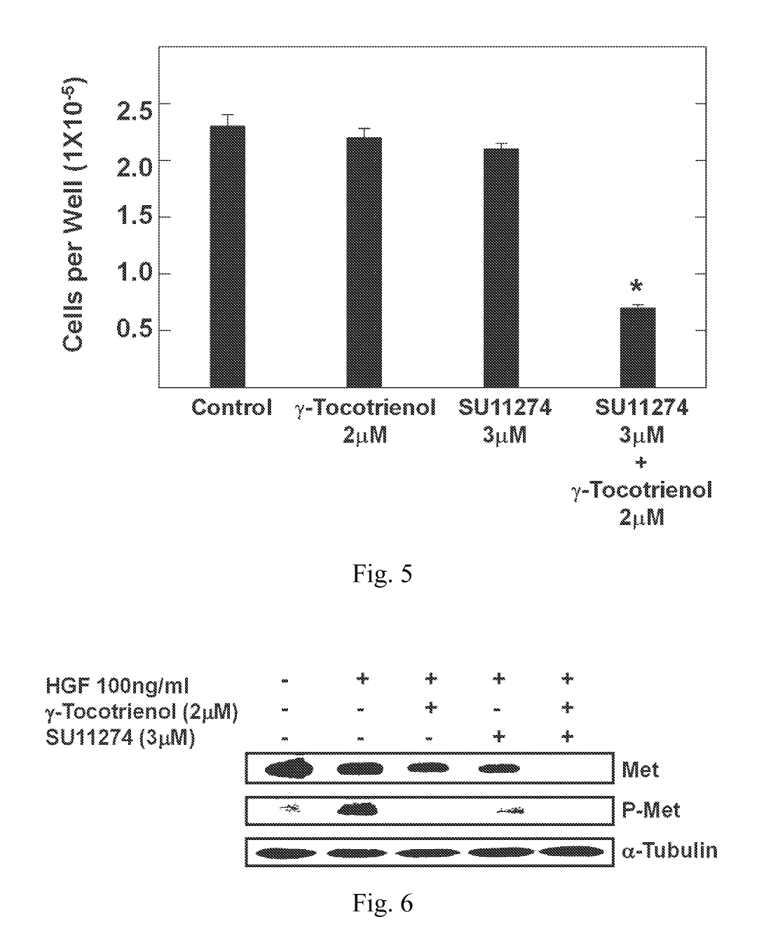

The effects of combined subeffective doses γ-tocotrienol with SU11274 on HGF-dependent growth of +SA mammary tumor cells are shown in FIG. 5. Treatment with subeffective doses of γ-tocotrienol (2 μM) or SU11274 (3 μM) alone had no effect on HGF-dependent +SA cell growth as compared to their respective vehicle-treated controls (FIG. 5). However, combined treatment with 2 μM γ-tocotrienol and 3 μM SU11274 resulted in a significant inhibition in +SA cell growth maintained in media containing 10 ng/mL HGF as compared to control cells and monotherapy treatment of each compound (FIG. 5).

+SA cells were initially plated at a density of 5×10⁴ cells/well (6 replicates/group) in 24-well plates and treated with 2 μM γ-tocotrienol or 3 μM SU11274 alone or in combination in the presence of 10 ng/mL of HGF as the mitogen for a 3-day culture period. Viable cell number was determined by the MTT colorimetric assay. Vertical bars indicates the mean viable cell number±SEM in each treatment group. A difference of P<0.05 was considered statistically significant as compared to the vehicle-treated control.

### Example 6

### Combined Treatment on Met

Further Western blot studies showed that treatment with a combination low-dose of SU11274 (3 μM) and γ-tocotrienol (2 μM) resulted in a large relative reduction in the expression of Met protein associated with a reduction in the phosphorylated (activated) levels of Met receptors as compared to cells in their respective vehicle-treated control groups and to individual subeffective treatments of SU11274 and γ-tocotrienol (FIG. 6).

FIG. 6 shows the effects of SU11274 or γ-tocotrienol treatment on HGF-dependent expression and tyrosine phosphorylation (activation) of Met in +SA mammary tumor cells. +SA cells were plated at a density of 1×10⁶ cells/100 mm culture dish. Cells were then incubated with serum-free media containing vehicle (control), 3 μM SU11274, 2 μM γ-tocotrienol or the combination of 3 μM SU11274 and 2 μM γ-tocotrienol, and 10 ng/mL HGF as a mitogen for a 3-day culture period. Following treatment exposure, cells were incubated in mitogen-free media overnight. Afterwards, cells were stimulated with 100 ng/mL HGF for 30 min Cells incubated with mitogen-free media were used as vehicle-treated controls. Following treatment exposure, whole cell lysates were prepared and then subjected to polyacrylamide gel electrophoresis and Western blot analysis for the visualization of total and phosphorylated (active) Met receptor levels. α-Tubulin was visualized to ensure equal sample loading in each lane. Each Western blot is a representative image of the data obtained for experiments that were repeated at least three times.

### Further Examples

In a series of embodiments based on the experiments described above, example compositions comprising micro-molar concentrations of γ-tocotrienol and micro-molar concentrations of SU11274 are presented as Examples 7-31 in Tables I, II, III, IV, and V which follow.

TABLE I

| μM | | | | | |
|---|---|---|---|---|---|
| concen- | Example | Example | Example | Example | Example |
| tration | 7 | 8 | 9 | 10 | 11 |
| γ-Toco- | 1-3 | 1-5 | 1-10 | 3-8 | 5-10 |
| trienol | | | | | |
| SU11274 | 1-3 | 1-3 | 1-3 | 1-3 | 1-3 |

TABLE II

| μM | | | | | |
|---|---|---|---|---|---|
| concen- | Example | Example | Example | Example | Example |
| tration | 12 | 13 | 14 | 15 | 16 |
| γ-Toco- | 1-3 | 1-5 | 1-10 | 3-8 | 5-10 |
| trienol | | | | | |
| SU11274 | 1-5 | 1-5 | 1-5 | 1-5 | 1-5 |

TABLE III

| μM | | | | | |
|---|---|---|---|---|---|
| concen- | Example | Example | Example | Example | Example |
| tration | 17 | 18 | 19 | 20 | 21 |
| γ-Toco- | 1-3 | 1-5 | 1-10 | 3-8 | 5-10 |
| trienol | | | | | |
| SU11274 | 1-10 | 1-10 | 1-10 | 1-10 | 1-10 |

TABLE IV

| μM | | | | | |
|---|---|---|---|---|---|
| concen- | Example | Example | Example | Example | Example |
| tration | 22 | 23 | 24 | 25 | 26 |
| γ-Toco- | 1-3 | 1-5 | 1-10 | 3-8 | 5-10 |
| trienol | | | | | |
| SU11274 | 3-8 | 3-8 | 3-8 | 3-8 | 3-8 |

TABLE V

| μM | | | | | |
|---|---|---|---|---|---|
| concen- | Example | Example | Example | Example | Example |
| tration | 27 | 28 | 29 | 30 | 31 |
| γ-Toco- | 1-3 | 1-5 | 1-10 | 3-8 | 5-10 |
| trienol | | | | | |
| SU11274 | 5-10 | 5-10 | 5-10 | 5-10 | 5-10 |

In a series of separate but related prophetic embodiments based on the experiments described above, example compositions comprising micro-molar concentrations of γ-tocotrienol and micro-molar concentrations of K252a are presented in Table VI.

TABLE VI

| μM | | | | | |
|---|---|---|---|---|---|
| concen- | Example | Example | Example | Example | Example |
| tration | 32 | 33 | 34 | 35 | 36 |
| γ-Toco- | 1-5 | 1-5 | 5-10 | 5-10 | 3-8 |
| trienol | | | | | |
| K252a | 1-5 | 5-10 | 1-5 | 5-10 | 3-8 |

In a series of separate but related prophetic embodiments based on the experiments described above, example compositions comprising micro-molar concentrations of γ-tocotrienol and micro-molar concentrations of PHA-665752 are presented in Table VII.

TABLE VII

| μM | | | | | |
|---|---|---|---|---|---|
| concen- | Example | Example | Example | Example | Example |
| tration | 37 | 38 | 39 | 40 | 41 |
| γ-Toco- | 1-5 | 1-5 | 5-10 | 5-10 | 3-8 |
| trienol | | | | | |
| PHA-665752 | 1-5 | 5-10 | 1-5 | 5-10 | 3-8 |

In a series of separate but related prophetic embodiments based on the experiments described above, example compositions comprising micro-molar concentrations of γ-tocotrienol and micro-molar concentrations of ARQ197 are presented in Table VIII.

TABLE VIII

| μM | | | | | |
|---|---|---|---|---|---|
| concen- | Example | Example | Example | Example | Example |
| tration | 42 | 43 | 44 | 45 | 46 |
| γ-Toco- | 1-5 | 1-5 | 5-10 | 5-10 | 3-8 |
| trienol | | | | | |
| ARQ197 | 1-5 | 5-10 | 1-5 | 5-10 | 3-8 |

In a series of separate but related prophetic embodiments based on the experiments described above, example compositions comprising micro-molar concentrations of γ-tocotrienol and micro-molar concentrations of XL880 are presented in Table IX.

TABLE IX

| μM | | | | | |
|---|---|---|---|---|---|
| concen- | Example | Example | Example | Example | Example |
| tration | 47 | 48 | 49 | 50 | 51 |
| γ-Toco- | 1-5 | 1-5 | 5-10 | 5-10 | 3-8 |
| trienol | | | | | |
| XL880 | 1-5 | 5-10 | 1-5 | 5-10 | 3-8 |

In a series of separate but related prophetic embodiments based on the experiments described above, example compositions comprising micro-molar concentrations of γ-tocotrienol and micro-molar concentrations of SGX523 are presented in Table X.

TABLE X

| μM | | | | | |
|---|---|---|---|---|---|
| concen- | Example | Example | Example | Example | Example |
| tration | 52 | 53 | 54 | 55 | 56 |
| γ-Toco- | 1-5 | 1-5 | 5-10 | 5-10 | 3-8 |
| trienol | | | | | |
| SGX523 | 1-5 | 5-10 | 1-5 | 5-10 | 3-8 |

In a series of separate but related prophetic embodiments based on the experiments described above, example compositions comprising micro-molar concentrations of γ-tocotrienol and micro-molar concentrations of MP470 are presented in Table XI.

TABLE XI

| μM | | | | | |
|---|---|---|---|---|---|
| concen- | Example | Example | Example | Example | Example |
| tration | 52 | 53 | 54 | 55 | 56 |
| γ-Toco- | 1-5 | 1-5 | 5-10 | 5-10 | 3-8 |
| trienol | | | | | |
| MP470 | 1-5 | 5-10 | 1-5 | 5-10 | 3-8 |

In a series of separate but related embodiments, any one of Examples 7-56 presented in Tables I-XI could be altered such that the γ-tocotrienol is replaced with an equivalent concentration of tocotrienol generally, i.e. not necessarily in the form of γ-tocotrienol.

All reagents were purchased from Sigma Chemical Company (St. Louis, Mo., USA) unless otherwise stated. Purified γ-tocotrienol was generously provided by First Tech International Ltd., (Hong Kong). Antibodies for Met (Cat #3127) and phospho-Met (Cat #3077) were purchased from Cell Signaling Technology (Beverly, Mass., USA) and these antibodies have been validated by their manufactures not to cross react with EGF receptors. Antibody for α-tubulin was purchased from EMD Biosciences (LA Jolla, Calif., USA). Antibody for phospho-Met was purchased from Santa Cruz Biotechnology (Santa Cruz, Calif., USA). Antibodies for EGF receptor, phospho-EGF receptor, cleaved caspase-3, and cleaved PARP were purchased from Cell Signaling Technology (Beverly, Mass., USA). Goat anti-rabbit and anti-mouse secondary antibodies were purchased from PerkinElmer Biosciences (Boston, Mass., USA). Alexa Fluor 594-conjugated anti-goat antibody, EGF was purchased from Invitrogen Corporation (Carlsbad, Calif., USA). HGF was purchased from PeproTech Inc., (Rocky Hill, N.J., USA).

Not wishing to be bound by theory, the anticancer effects of γ-tocotrienol appears to be mediated, at least in part, through the suppression of Met expression and activation. Treatment with γ-tocotrienol or SU11274, a specific inhibitor of Met receptor tyrosine kinase activity, inhibited HGF-dependent Met tyrosine phosphorylation and +SA tumor cell growth in a dose-responsive manner. Further, γ-tocotrienol appears to be a potent inhibitor of Met receptor tyrosine kinase activity and can effectively inhibit HGF-dependent +SA cell growth. Combined treatment of γ-tocotrienol with other receptor tyrosine kinase inhibitors may provide significantly more potent anticancer effectiveness than that observed with each treatment alone.

In a related embodiment, γ-tocotrienol may be combined with other chemotherapeutic agents in the treatment of breast cancer in women characterized by amplified or deregulated Met mitogenic signaling.

The compositions disclosed herein may be delivered intravenously, intraperitoneally, subcutaneously, intramuscularly, ocularly, orally, transdermally, topically, by inhalation or by other suitable means.

Individual compositions disclosed herein may be used in the treatment of one or more forms of cancer and may be used to treat a cancer selected from leukemia, non-small cell lung cancer, colon cancer, central nervous system cancer, melanoma, ovarian cancer, renal cancer, prostate cancer, and breast cancer.

As used herein, the term “pharmaceutically acceptable salt” refers to a salt prepared from any one or multiple non-toxic acid(s) or base(s), including both organic and inorganic acids and bases that are suitable for use in contact with living animal or human tissue without causing adverse physiological responses.

Any and all reference to patents, documents and other writings contained herein shall not be construed as an admission as to their status with respect to being or not being prior art. It is understood that the array of features and embodiments taught herein may be combined and rearranged in a large number of additional combinations not directly disclosed, as will be apparent to one having skill in the art and that various embodiments of the invention may have less than all of the benefits and advantages disclosed herein.

There are, of course, other alternate embodiments which are obvious from the foregoing descriptions, which are intended to be included within the scope of the invention, as defined by the following claims.

I claim:

1. A composition of matter comprising γ-tocotrienol and SU11274.

2. The composition of matter of claim 1 wherein γ-tocotrienol is present in a concentration of 2.0 μM or greater.

3. The composition of matter of claim 1 wherein SU11274 is present in a concentration of 3.0 μM or greater.

4. A composition of matter comprising a tocotrienol and a Met kinase inhibitor.

5. The composition of matter of claim 4 wherein the tocotrienol is γ-tocotrienol.

6. The composition of matter of claim 4 wherein the Met kinase inhibitor is SU11274.

7. The composition of matter of claim 4 wherein the tocotrienol is present in a concentration of 2.0 μM or greater.

8. The composition of matter of claim 4 wherein the Met kinase inhibitor is present in a concentration of 3.0 μM or greater.

9. The composition of matter of claim 4 wherein the tocotrienol is present in a concentration of 3.0 μM or less.

10. The composition of matter of claim 4 wherein the Met kinase inhibitor is present in a concentration of 4.0 μM or less.

11. A composition of matter comprising:
(a) a compound selected from γ-tocotrienol and a pharmaceutically acceptable salt of γ-tocotrienol and
(b) a compound selected from SU11274 and a pharmaceutically acceptable salt of SU11274.

12. A composition of matter comprising a tocotrienol and a compound selected from K252a, SU11274, PHA-665752, ARQ197, XL880, SGX523, and MP470.

13. The composition of claim 12 wherein the tocotrienol is γ-tocotrienol.

14. The composition of matter of claim 12 wherein the compound selected from K252a, SU11274, PHA-665752, ARQ197, XL880, SGX523, and MP470 has a concentration between 1 μM and 5 μM.

15. The composition of matter of claim 12 wherein the compound selected from K252a, SU11274, PHA-665752, ARQ197, XL880, SGX523, and MP470 a concentration between 5 μM and 10 μM.

16. The composition of matter of claim 12 wherein the compound selected from K252a, SU11274, PHA-665752, ARQ197, XL880, SGX523, and MP470 has a concentration between 3 μM and 8 μM.

17. The composition of matter of claim 12 wherein the tocotrienol has a concentration between 1 μM and 5 μM.

18. The composition of matter of claim 12 wherein the tocotrienol has a concentration between 5 μM and 10 μM.

19. The composition of matter of claim 12 wherein the tocotrienol has a concentration between 3 μM and 8 μM.
